(19) United States
(12) Patent Application Publication
(10) Pub. No.: US 2018/0243439 A1
(43) Pub. Date: Aug. 30, 2018

(54) Pharmaceutical Formulation of an Anti-Guanylyl Cyclase C Antibody Conjugate Comprising Histidine or a Salt Thereof and Polysorbate 20

(71) Applicant: MILLENNIUM PHARMACEUTICALS, INC., Cambridge, MA (US)

(72) Inventors: Shohei HORIUCHI, Osaka-shi, Osaka (JP); Tomomi SATO, Osaka-shi, Osaka (JP)

(73) Assignee: MILLENNIUM PHARMACEUTICALS, INC., Cambridge, MA (US)

(21) Appl. No.: 15/305,779

(22) PCT Filed: Apr. 23, 2015
(86) PCT No.: PCT/US2015/027255

§ 371 Date: Oct. 21, 2016

(30) Foreign Application Priority Data
Apr. 24, 2014 (JP) 2014-090602

(51) Int. Cl. A61K 47/68; C07K 16/40; A61K 47/26; A61K 47/22; A61K 9/00; A61K 9/19

(52) U.S. Cl. CPC A61K 47/6871; C07K 16/40; A61K 47/6889; A61K 47/6811; C07K 2317/565; A61K 47/22; A61K 9/0019; A61K 9/19; A61K 47/26

(57) ABSTRACT

Provided is a formulation containing an immunoconjugate comprising an anti-GCC antibody molecule and a therapeutic agent, which is superior in stability and useful as a prophylactic or therapeutic agent for gastrointestinal cancer and the like. A formulation comprising (i) an immunoconjugate of the following formula (I): wherein Ab is an anti-GCC antibody molecule, X is a linker component, Z is a therapeutic agent, and m is an integer from 1-15, or a pharmaceutically acceptable salt thereof, (ii) polysorbate 20, and (iii) histidine or a salt thereof.

### TECHNICAL FIELD

The present invention relates to a formulation containing an immunoconjugate comprising an anti-GCC antibody molecule and a therapeutic agent.

### BACKGROUND OF THE INVENTION

Guanylyl cyclase C (sometimes to be abbreviated as GCC in the present specification) is a transmembrane cell surface receptor expressed in mucosal cells that line the small intestine, large intestine and rectum, which functions for the maintenance of the intestinal fluid, homeostasis of electrolyte, and cell proliferation (non-patent documents 1 and 2). It is known that the expression of GCC is maintained in neoplastic transformation of intestinal epithelial cells (non-patent documents 2-4).

As an anti-GCC antibody and an immunoconjugate thereof, those of patent document 1 are known.

### DOCUMENT LIST

### Patent Document

[patent document 1] WO 2011/050242

### Non-Patent Document

[non-patent document 1] Carrithers et al., Proc. Natl. Acad. Sci. USA vol. 100: pages 3018-3020 (2003)
[non-patent document 2] Carrithers et al., Dis Colon Rectum vol. 39: pages 171-181 (1996)
[non-patent document 3] Buc et al., Eur J Cancer vol. 41: pages 1618-1627 (2005)
[non-patent document 4] Carrithers et al., Gastroenterology vol. 107: pages 1653-1661 (1994)

### SUMMARY OF THE INVENTION

### Problems to be Solved by the Invention

An object of the present invention is to provide a formulation containing an immunoconjugate comprising an anti-GCC antibody molecule and a therapeutic agent, which is superior in stability and useful as a prophylactic or therapeutic agent for gastrointestinal cancer and the like.

### Means of Solving the Problems

The present inventors have conducted intensive studies of a formulation containing an immunoconjugate represented by the following formula (I), and found that a formulation superior in stability can be obtained by adding polysorbate 20 and histidine or a salt thereof and the like in addition to the above-mentioned immunoconjugate, which resulted in the completion of the present invention.

Accordingly, the present invention relates to [1] a formulation comprising

(i) an immunoconjugate of the following formula (I):

AbX-X)ₘ (I)

wherein
Ab is an anti-GCC antibody molecule,
X is a linker component,
Z is a therapeutic agent, and
m is an integer from 1-15,
or a pharmaceutically acceptable salt thereof,
(ii) polysorbate 20, and
(iii) histidine or a salt thereof
(sometimes to be abbreviated as “the formulation of the present invention” in the present specification);
[2] the formulation of [1], comprising 0.08% (w/v) of polysorbate 20;
[3] the formulation of [1] or [2], comprising 10 mM of histidine or a salt thereof;
[4] the formulation of any of [1]-[3], further comprising a saccharide;
[5] the formulation of [4], wherein the saccharide is a non-reducing sugar;
[6] the formulation of [4], wherein the saccharide is sucrose;
[7] the formulation of [4], comprising 7.5% (w/v) of saccharide;
[8] the formulation of any of [1]-[7], comprising 25 mg/ml of the immunoconjugate;
[9] the formulation of any of [1]-[8], which has a pH within the range of 4.9-5.5;
[10] the formulation of any of [1]-[9], wherein the anti-GCC antibody molecule comprises complementarity determining regions defined by the following amino acid sequences,
light chain: CDR1 SEQ ID NO: 1
CDR2 SEQ ID NO: 2
CDR3 SEQ ID NO: 3
   heavy chain: CDR1 SEQ ID NO: 4
CDR2 SEQ ID NO: 5
CDR3 SEQ ID NO: 6;
   [11] the formulation of any of [1]-[9], wherein the anti-GCC antibody molecule comprises a light chain variable region defined by the amino acid sequence of SEQ ID NO: 7 and a heavy chain variable region defined by the amino acid sequence of SEQ ID NO: 8;
   [12] the formulation of any of [1]-[11],
   wherein X is -Ap-Wq-Yr-
   wherein
   A is a stretcher unit,
   p is 0 or 1,
   each W is independently an amino acid unit,
   q is an integer from 0-12,
   Y is a self-immolative spacer unit, and
   r is an integer from 0-2;
   [13] the formulation of any of [1]-[12], wherein Z is maytansine or auristatin;
   [14] the formulation of any of [1]-[13], wherein Z is monomethylauristatin E;
   [15] the formulation of any of [1]-[14], wherein m is an integer from 3-5;
   [16] the formulation of any of [1]-[14], wherein the immunoconjugate is represented by the formula (I-5):

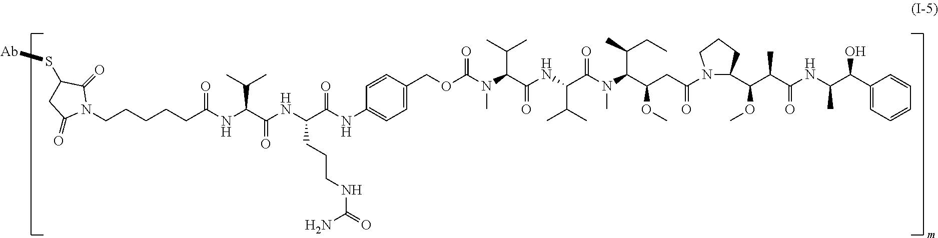

wherein
Ab is an anti-GCC antibody molecule, and
m is an integer from 1-15;
[17] the formulation of any of [1]-[16], which is an injection formulation;
[18] the formulation of [17], which is a formulation for intravenous injection;
[19] the formulation of any of [1]-[18], which is a liquid formulation;
[20] the formulation of any of [1]-[18], which is a frozen liquid formulation;
[21] the formulation of any of [1]-[18], which is a freeze-dry formulation;
[22] a formulation comprising
(i) an immunoconjugate represented by the following formula (I-5):

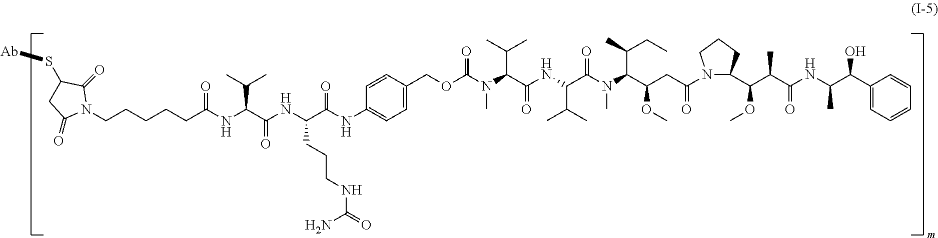

wherein
Ab is an anti-GCC antibody molecule comprising a complementarity determining region defined by the following amino acid sequence:
light chain: CDR1 SEQ ID NO: 1
CDR2 SEQ ID NO: 2
CDR3 SEQ ID NO: 3
   heavy chain: CDR1 SEQ ID NO: 4
CDR2 SEQ ID NO: 5
CDR3 SEQ ID NO: 6, and
   m is an integer from 1-8,
   or a pharmaceutically acceptable salt thereof,
   (ii) 0.08% (w/v) of polysorbate 20,
   (iii) 10 mM of histidine or a salt thereof, and
   (iv) 7.5% (w/v) of sucrose,
   which has a pH within the range of 4.9-5.5;
   and the like.

The present invention further provides the formulation below:

[23] the formulation of [22], wherein m is an integer from 3-5, preferably 4;
[24] the formulation of any of [1]-[14], wherein the formulation comprises a plurality of immunoconjugates of formula (I), wherein m is the average number of —X—Z moieties per anti-GCC antibody molecule;
[25] the formulation of [24], wherein m is the integer 4;
[26] the formulation of any of [1]-[14], wherein the formulation comprises a plurality of immunoconjguates of formula (I), and wherein two or more of the immunoconjugates have a different m value;
[27] the formulation of any of [16]-[22], wherein the formulation comprises a plurality of immunoconjugates of formula (I-5), wherein m is the average number of —X—Z moieties per anti-GCC antibody molecule;
[28] the formulation of [27], wherein m is the integer 4;
[29] the formulation of any of [16]-[22], wherein the formulation comprises a plurality of immunoconjugates of formula (I-5), and wherein two of more of the immunoconjugates have a different m value.

### Effect of the Invention

According to the present invention, a formulation superior in stability, which contains the above-mentioned immunoconjugate, is provided. To be specific, a medicament superior in stability is provided by adding polysorbate 20 and histidine or a salt thereof and the like in addition to the aforementioned immunoconjugate, since the development of a complex of the immunoconjugate (sometimes to be referred simply to a complex in the present specification) is suppressed. According to the present invention, moreover, a medicament superior in stability is provided since the immunoconjugate exists stably as a monomer in the formulation. According to the present invention, moreover, a medicament superior in stability, wherein the development of turbidness in a solution formulation in the presence of a stress is suppressed, is provided.

### DETAILED DESCRIPTION OF THE INVENTION

In the present specification, unless otherwise defined, the scientific terms and technical terms used in relation to the present invention have meanings commonly understood by those of ordinary skill in the pertinent technical field. For example, the techniques used in relation to the cell culture and tissue culture; molecular biological method (e.g., DNA recombination, tissue culture, and transfection (e.g., electroporation, lipofection)); synthesis of protein, oligonucleotide or polynucleotide; and hybridization described in the present specification can be performed by the general methods described in the documents known in the pertinent technical field (e.g., Molecular Cloning by Sambrook et al.: A Laboratory Manual (Cold Spring Harbor Laboratory Press, 3rd edition, Cold Spring Harbor, N.Y. (2000)); Antibodies by Harlow, E. and Lane, D. (1988); A Laboratory Manual, Cold Spring Harbor Laboratory Press, Cold Spring Harbor, N.Y.), the method described in patent document 1 or a method analogous to those methods. In addition, the enzyme reactions and purification techniques can be performed according to the methods known per se or explanations of manufacturers. The nomenclature method, test method and techniques utilized for the analytical chemistry, organic synthetic chemistry, as well as medicinal chemistry and pharmacochemistry described in the present specification are also known in the pertinent technical field. GenBank accession number and GenPept accession number can be found at the websites owned by National Center for Biotechnological Information, Bethesda Md.

In the present specification, examples of the “halogen” include fluorine, chlorine, bromine and iodine.

In the present specification, examples of the “C₁-C₈ alkyl” include methyl, ethyl, n-propyl, isopropyl, n-butyl, iso-butyl, sec-butyl, tert-butyl, n-pentyl, 2-pentyl, 3-pentyl, 2-methyl-2-butyl, n-hexyl, n-heptyl, n-octyl, n-nonyl, n-decyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl.

In the present specification, examples of the “C₂-C₈ alkenyl” include ethenyl, allyl, 1-butenyl, 2-butenyl, isobutylenyl, 1-pentenyl, 2-pentenyl, 3-methyl-1-butenyl, 2-methyl-2-butenyl and 2,3-dimethyl-2-butenyl.

In the present specification, examples of the “C₂-C₈ alkynyl” include propargyl, acetylenyl, propynyl, 1-butynyl, 2-butynyl, 1-pentynyl, 2-pentynyl and 3-methyl-1butynyl.

In the present specification, the “arylene” is C₆-C₁₄ arylene. Examples of the C₆-C₁₄ arylene include phenylene, naphthylene and anthrylene. When the arylene is phenylene, for example, it can take the ortho, meta or para configuration as shown in the following structure.

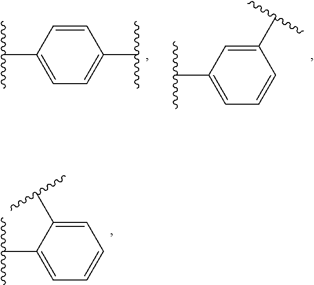

In the present specification, examples of the “C₁-C₁₀ alkylene” include methylene, ethylene, propylene, butylene, pentylene, hexylene, heptylene, octylene, nonylene, decalene and 1,4-cyclohexylene.

The immunoconjugate used in the present invention is represented by formula (I):

AbX-Z)ₘ (I)

as described in patent document 1, the contents of which are incorporated by reference herein in its entirety, and is described in further detail below.

The anti-GCC antibody molecule for Ab is an anti-GCC antibody molecule described in patent document 1. As the anti-GCC antibody molecule in the present specification, a monoclonal antibody is preferable. From another aspect, moreover, as the anti-GCC antibody molecule in the present specification, a complete human antibody molecule is preferable. From still another aspect, as the anti-GCC antibody molecule in the present specification, IgG is preferable, and IgG1 is particularly preferable.

As the anti-GCC antibody molecule, an anti-GCC antibody molecule or a derivative antibody molecule containing the anti-GCC antibody molecule as a reference antibody molecule, which contains the complementarity determining regions (CDR) defined by the following amino acid sequences (particularly, anti-GCC antibody molecule containing the following CDR) is preferable:

light chain: CDR1 SEQ ID NO: 1
CDR2 SEQ ID NO: 2
CDR3 SEQ ID NO: 3
   heavy chain: CDR1 SEQ ID NO: 4
CDR2 SEQ ID NO: 5
CDR3 SEQ ID NO: 6

As the anti-GCC antibody molecule, an anti-GCC antibody molecule or a derivative antibody molecule containing the anti-GCC antibody molecule as a reference antibody molecule, which contains the variable regions defined by the following amino acid sequences (particularly, anti-GCC antibody molecule containing the following variable regions) is more preferable:

light chain: variable region SEQ ID NO: 7
heavy chain: variable region SEQ ID NO: 8

As the anti-GCC antibody molecule in the present specification, an anti-GCC antibody molecule or a derivative antibody molecule containing the anti-GCC antibody molecule as a reference antibody molecule, which contains the light chain and heavy chain defined by the following amino acid sequences is further preferable and, among others, an anti-GCC antibody molecule containing the light chain and heavy chain defined below is preferable:

light chain: SEQ ID NO: 9
heavy chain: SEQ ID NO: 10

As the anti-GCC antibody molecule in the present specification, antibody 5F9 (sometimes to be referred to as 5F9 or 5F9 mAb in the present specification) is particularly preferable.

The antibody 5F9 can be produced by hybridoma 5F9 also called hybridoma 46.5F9.8.2. This was deposited under accession number PTA-8132 at the American Type Culture Collection, 10801 University Boulevard, Manassas, Va. 20110, U.S.A. on Jan. 10, 2007 by Millennium Pharmaceuticals Inc., 40 Landsdowne Street, Cambridge, Mass., 02139, USA (deposited under the Budapest Treaty on the International Recognition of the Deposit of Microorganisms for the Purposes of Patent Procedure and satisfies the requirements therefor).

In the present specification, the derivative antibody molecule refers to an antibody molecule having the amino acid sequence of a reference antibody molecule, which underwent conservative amino acid substitution or non-essential amino acid substitution within the range free from a substantial influence on the function of the reference antibody molecule (e.g., function to interact with GCC (e.g., human GCC), or function to recognize same (e.g., specifically bind to GCC (e.g., human GCC)).

Whether a particular conservative amino acid substitution or non-essential amino acid substitution is acceptable can be determined, for example, by the method described in Bowie, J U et al., Science vol. 247: pages 1306-1310 (1990) and Padlan et a., FASEB J. vol. 9: pages 133-139 (1995).

In the present specification, the “conservative amino acid substitution” means substitution of an amino acid residue by an amino acid residue having a similar side chain to said amino acid residue. The family of the amino acid residue having a similar side chain is generally known in the pertinent technical field. Examples of the above-mentioned family include basic side chain family (e.g., lysine, arginine, histidine), acidic side chain family (e.g., aspartic acid, glutamic acid), uncharged polar side chain family (e.g., asparagine, glutamine, serine, threonine, tyrosine, cysteine), nonpolar side chain family (e.g., glycine, alanine, valine, leucine, isoleucine, proline, phenylalanine, methionine, tryptophan), beta branched side chain family (e.g., threonine, valine, isoleucine), and aromatic side chain family (e.g., tyrosine, phenylalanine, tryptophan, histidine).

In the present specification, the “non-essential amino acid substitution” means substitution of an amino acid residue which does not accompany disappearance of the biological activity (e.g., functions recited as examples of the function of the aforementioned reference antibody molecules) or substantial change of the biological activity.

The “linker component” for X is the linker component described in patent document 1.

Examples of the linker component include -Ap-Wq-Yr- wherein A is a stretcher unit,

p is 0 or 1,
each W is independently an amino acid unit,
q is an integer from 0-12,
Y is a self-immolative spacer unit, and
r is an integer from 0-2.

The “stretcher unit” for A is the stretcher unit described in patent document 1.

Examples of the “stretcher unit” include the structures in the brackets in the following formula (IIa):

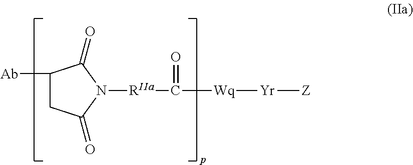

wherein R^{IIa} is —(CH₂)₅—, —(CH₂CH₂O)₂—CH₂—, -arylene- or -arylene-C₁-C₁₀ alkylene-,
the following formula (IIb):

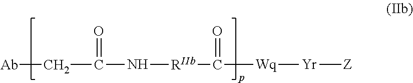

wherein R^{IIb} is —(CH₂)₅—, and
the following formula (IIc):

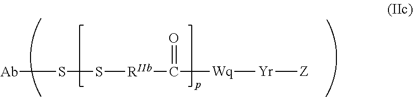

wherein R^{IIc} is —(CH₂)₅—, —(CH₂CH₂O)₂—CH₂—, -arylene- or -arylene-C₁-C₁₀ alkylene-.

In the present specification, unless otherwise shown by the context, component S in the following formula is sulfur atom in unit Ab (that is, anti-GCC antibody).

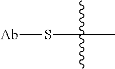

As the “stretcher unit”, the structure shown in the brackets in the formula (IIa) wherein R^{IIa} is —(CH₂)₅— is preferable.

p is preferably 1.

The “amino acid unit” for W is the amino acid unit described in patent document 1. When the linker component contains a plurality of “amino acid units” for W, which means that q is an integer from 2-12, the respective “amino acid units” may be the same or different.

q is preferably an integer from 0-5, more preferably 2.

Examples of -Wq- include valine-citrulline, phenylalanine-lysine, N-methylvaline-citrulline, 5-aminovaleric acid, homophenylalanine lysine, tetraisoquinolinecarboxylate lysine, cyclohexylalanine lysine, isonepecotic acid lysine, beta-alanine lysine, glycine serine valine glutamine and isonepecotic acid.

As the -Wq-, valine-citrulline is preferable.

The “self-immolative spacer unit” for Y is the self-immolative spacer unit described in patent document 1.

Examples of the “self-immolative spacer unit” include

(1) p-aminobenzylalcohol wherein the phenylene moiety is optionally substituted by 1 to 3 substituents selected from —C₁-C₈ alkyl, —C₂-C₈ alkenyl, —C₂-C₈ alkynyl, —O—(C₁-C₈ alkyl), —O—(C₂-C₈ alkenyl), —O—(C₂-C₈ alkynyl), -halogen, -nitro and -cyano, and
(2) p-aminobenzene wherein the phenylene moiety is optionally substituted by 1 to 3 substituents selected from —C₁-C₆ alkyl, —C₂-C₈ alkenyl, —C₂-C₈ alkynyl, —O—(C₁-C₈ alkyl), —O—(C₂-C₈ alkenyl), —O—(C₂-C₈ alkynyl), -halogen, -nitro and -cyano.

As the “self-immolative spacer unit”, p-aminobenzylalcohol is preferable.

As r, 1 or 2 is preferable, and 1 is more preferable.

Specific examples of the linker component include maleimidocaproyl (mc); maleimidocaproyl-p-aminobenzylcarbamate; maleimidocaproyl-peptide-aminobenzylcarbamate (e.g., maleimidocaproyl-L-phenylalanine-L-lysin-p-aminobenzylcarbamate and maleimidocaproyl-L-valine-L-citrulline-p-aminobenzylcarbamate (vc)); N-succinimidyl 3-(2-pyridyldithio)proprionate (N-succinimidyl 4-(2-pyridyldithio)pentanoate; 4-succinimidyl-oxycarbonyl-2-methyl-2-(2-pyridyldithio)-toluene (SMPT); N-succinimidyl 3-(2-pyridyldithio)propionate (SPDP); N-succinimidyl 4-(2-pyridyldithio)butyrate (SPDB); 2-iminothiolane; S-acetylsuccinic anhydride; disulfidobenzylcarbamate; carbonate; hydrazone linker; N-(α-maleimidoacetoxy)succinimide ester; N-[4-(p-azidosalicylamido)butyl]-3′-(2′-pyridyldithio)propionamide (AMAS); N-[β-maleimidopropyloxy]succinimide ester (BMPS); [N-ε-maleimidocaproyloxy]succinimide ester (EMCS); N-[γ-maleimidobutyryloxy]succinimide ester (GMBS); succinimidyl-4-[N-maleimidomethyl]cyclohexane-1-carboxy-[6-amidocaproate] (LC-SMCC); succinimidyl 6-(3-[2-pyridyldithio]-propionamido)hexanoate (LC-SPDP); m-maleimidobenzoyl-N-hydroxysuccinimide ester (MBS); N-succinimidyl [4-iodoacetyl]aminobenzoate (STAB); succinimidyl 4-[N-maleimidomethyl]cyclohexane-1-carboxylate (SMCC); N-succinimidyl 3-[2-pyridyldithio]-propionamide (SPDP); [N-ε-maleimidocaproyloxy] sulfosuccinimide ester (sulfo-EMCS); N-[γ-maleimidobutyryloxy]sulfosuccinimide ester (sulfo-GMBS); 4-sulfosuccinimidyl-6-methyl-α-(2-pyridyldithio)toluamido]hexanoate) (sulfo-LC-SMPT); sulfosuccinimidyl 6-(3′-[2-pyridyldithio]-propionamido)hexanoate (sulfo-LC-SPDP); m-maleimidobenzoyl-N-hydroxysulfosuccinimide ester (sulfo-MBS); N-sulfosuccinimidyl [4-iodoacetyl]aminobenzoate (sulfo-SIAB); sulfosuccinimidyl 4-[N-maleimidomethyl]cyclohexane-1-carboxylate (sulfo-SMCC); sulfosuccinimidyl 4-[p-maleimidophenyl]butyrate (sulfo-SMPB); ethylene glycol-bis(succinic acid N-hydroxysuccinimide ester) (EGS); disuccinimidyl tartrate (DST); 1,4,7,10-tetraazacyclododecane-1,4,7,10-tetraacetic acid (DOTA); diethylenetriamine-pentaacetic acid (DTPA); and thiourea linker.

As the linker component, maleimidocaproyl-peptide-aminobenzylcarbamate is preferable, and maleimidocaproyl-L-valine-L-citrulline-p-aminobenzylcarbamate (sometimes referred to as vc in the present specification) is more preferable.

The “therapeutic agent” for Z is the therapeutic agent described in patent document 1.

Examples of the “therapeutic agent” include anti-cancer agents and chemotherapeutic agents (e.g., a reactant that inhibits the onset or progression of neoplasm in human (particularly, lesions such as carcinoma, sarcoma, lymphoma, leukemia and the like); a reactant that inhibits metastasis of neoplasm or neovascularization; a cytotoxic agent; a cell proliferation inhibitor (reactant that inhibits or suppresses cell proliferation and/or cell proliferation)).

Examples of the cytotoxic agent or cell proliferation inhibitor include metabolic antagonist (e.g., azathiopurine, 6-mercaptopurine, 6-thioguanine, fludarabine, pentostatin, cladribine, 5-fluorouracil (5FU), floxuridine (FUDR), cytosine arabinoside (cytarabine), methotrexate, trimethoprim, pyrimethamine, pemetrexed); alkylating agent (e.g., cyclophosphamide, mechlorethamine, uramustine, melphalan, chlorambucil, thiotepa/chlorambucil, ifosfamide, carmustine, lomustine, streptozocin, busulfan, dibromomannitol, cisplatin, carboplatin, nedaplatin, oxaliplatin, satraplatin, triplatin tetranitrate, procarbazine, altretamine, dacarbazine, mitozolomide, temozolomide); anthracycline (e.g., daunorubicin, doxorubicin, epirubicin, idarubicin, valrubicin); antibiotic (e.g., dactinomycin, bleomycin, mithramycin, anthramycin, streptozotocin, gramicidin D, mitomycins (e.g., mitomycin C), duocarmycins (e.g., CC-1065), calicheamicins); mitotic inhibitor (maytansinoids (e.g., maytansine), auristatin (e.g., auristatin E, auristatin phenylalanine phenylenediamine (AFP), monomethylauristatin E, and monomethylauristatin F), dolastatins, cryptophycins, vinca alkaloid (e.g., vincristine, vinblastine, vindesine, vinorelbine), taxanes (e.g., paclitaxel, docetaxel, novel taxane (e.g., see WO 01/38318), and colchicines; topoisomerase inhibitor (e.g., irinotecan, topotecan, amsacrine, etoposide, teniposide, mitoxantrone); and proteasome inhibitor (e.g., peptidylboronic acid); or pharmaceutically acceptable salts thereof (specifically, salts of the same kind as those recited later as specific examples of the salts of histidine).

As the therapeutic agent, a mitotic inhibitor is preferable; maytansinoids or auristatin is more preferable; maytansine or auristatin (particularly, monomethylauristatin E) is further preferable; and monomethylauristatin E (sometimes to be referred to as MMAE in the present specification) is further more preferable.

The variable m represents the number of —X—Z moieties per anti-GCC antibody molecule in the immunoconjugate of formula (I). In various embodiments, m ranges from 1 to 15, 1 to 10, 1 to 9, 1 to 8, 1 to 7, 1 to 6, 1 to 5, 1 to 4, 1 to 3, or 1 to 2. In compositions comprising a plurality of immunoconjugates of formula (I), m is the average number of —X—Z moieties per Ab, also referred to as the average drug loading. Average drug loading may range from 1 to about 15 —X—Z moieties per Ab. In some embodiments, when m represents the average drug loading, m is about 1, about 2, about 3, about 4, about 5, about 6, about 7 or about 8. In exemplary embodiments, m is from about 1 to about 15, preferably from about 1 to about 8, more preferably from about 3 to about 5, even more preferably about 4.

The average number of —X—Z moieties per Ab may be characterized by conventional means such as mass spectrometry, ELISA assay, and HPLC. The quantitative distribution of immunoconjugates in terms of m may also be determined. In some instances, separation, purification, and characterization of homogenous immunoconjugates where m is a certain value, as distinguished from immunoconjugates with other drug loadings, may be achieved by means such as reverse phase HPLC or electrophoresis.

The immunoconjugate in the formulation of the invention may exist as mixtures of immunoconjugate components, wherein each immunoconjugate component of the mixture has a different m value. For example, an immunoconjugate of the formulation of the invention may exist as mixture of two or more separate immunoconjugate components, one immunoconjugate component wherein the m is 3, and the other immunoconjugate component wherein the m is 5.

In the present specification, the “immunoconjugate” is an antibody molecule (e.g., anti-GCC antibody molecule) conjugated with a nonantibody component (e.g., therapeutic agent).

In the present specification, the immunoconjugate is the immunoconjugate described in patent document 1.

As the immunoconjugate in the present invention, an immunoconjugate represented by the following formula (I-5)

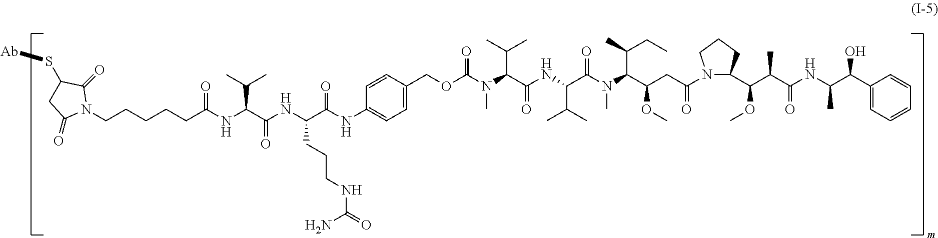

wherein Ab is an anti-GCC antibody molecule, and m is an integer from 1-15,
is preferable.

As the immunoconjugate in the present invention, an immunoconjugate represented by the following formula (I-5)

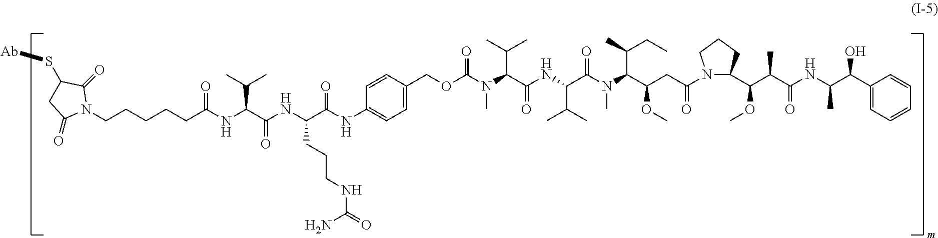

wherein Ab is an anti-GCC antibody molecule comprising a complementarity determining region defined by the following amino acid sequence:
light chain: CDR1 SEQ ID NO: 1
CDR2 SEQ ID NO: 2
CDR3 SEQ ID NO: 3
   heavy chain: CDR1 SEQ ID NO: 4
CDR2 SEQ ID NO: 5
CDR3 SEQ ID NO: 6, and
   m is an integer from 1-8,
   is more preferable.

As the immunoconjugate in the present invention, an immunoconjugate wherein the anti-GCC antibody molecule in the above-mentioned formula (I-5) is 5F9, and m is an integer from 3-5 is further more preferable.

In the immunoconjugate of the present invention, a bond formation between Ab, X and Z can be performed by a method known per se, methods described in known documents (e.g., Doronina et al., Nature Biotech., vol. 21: pages 778-784 (2003); Hamblett et al., Clin. Cancer Res., vol. 10: pages 7063-7070 (2004); Carter and Senter, Cancer J., 14 pages 154-169 (2008); U.S. Pat. Nos. 7,498,298, 7,091,186, 6,884,869; 6,323,315; 6,239,104; 6,034,065; 5,780,588; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,816,444; and 4,486,414; US-A-20090010945, 20060074008, 20080300192, 20050009751, 20050238649 and 20030083236; WO04/010957, WO02/088172 and WO2011/053686), or a method analogous thereto.

The immunoconjugate may be a pharmaceutically acceptable salt. Examples of such salt include salt with inorganic base, salt with organic base, salt with inorganic acid and salt with organic acid.

Preferable examples of the salt with inorganic base include alkali metal salts such as sodium salt, potassium salt and the like; alkaline earth metal salts such as calcium salt, magnesium salt and the like; aluminum salt; and ammonium salt.

Preferable examples of the salt with organic base include salts with trimethylamine, triethylamine, pyridine, picoline, ethanolamine, diethanolamine, triethanolamine, dicyclohexylamine, N,N-dibenzylethylenediamine and the like.

Preferable examples of the salt with inorganic acid include salts with hydrochloric acid, hydrobromic acid, nitric acid, sulfuric acid, phosphoric acid and the like.

Preferable examples of the salt with organic acid include salts with formic acid, acetic acid, trifluoroacetic acid, fumaric acid, oxalic acid, tartaric acid, maleic acid, citric acid, succinic acid, malic acid, methanesulfonic acid, benzenesulfonic acid, p-toluenesulfonic acid and the like.

The content of the immunoconjugate or a salt thereof in the formulation of the present invention is preferably 0.1-20 wt %, more preferably 0.8-6 wt %, further preferably 1.5-3.0 wt %, further more preferably 2.5 wt %, relative to the total amount of the formulation.

When the formulation of the present invention is a liquid formulation, the concentration of the immunoconjugate or a salt thereof in the liquid formulation is preferably 1-200 mg/ml, more preferably 8-60 mg/ml, further preferably 15-30 mg/ml, and further more preferably 25 mg/ml.

The formulation of the present invention contains (ii) polysorbate 20, whereby the stability of the immunoconjugate in the formulation can be improved (formation of an immunoconjugate complex is suppressed, an increase in the turbidity under mechanical stress is suppressed, etc.). Polysorbate 40 or polysorbate 60 can also be used instead of polysorbate 20.

As the formulation of the present invention, a formulation containing not less than 0.03 wt % of polysorbate 20 relative to the total amount of the formulation is preferable, since the stability against the stress (e.g., mechanical stress) of the immunoconjugate increases.

The content of the polysorbate 20 in the formulation of the present invention is preferably 0.03-1 wt %, more preferably 0.04-0.5 wt %, further preferably 0.06-0.1 wt %, and further more preferably 0.08 wt %, relative to the total amount of the formulation.

When the formulation of the present invention is a liquid formulation, the content of polysorbate 20 in the liquid formulation is preferably 0.03-1% (w/v), more preferably 0.04-0.5% (w/v), further preferably 0.06-0.1% (w/v), further more preferably 0.08% (w/v).

The formulation of the present invention contains (iii) histidine or a salt thereof, whereby the stability of the immunoconjugate in the formulation is improved. Phosphoric acid or a salt thereof can also be used instead of histidine or a salt thereof.

As the salt of histidine, a pharmaceutically acceptable salt is preferable. Examples of such salt include salts with inorganic base and salts with organic base, from the salts the aforementioned immunoconjugate can form. As the salt of histidine, hydrochloride is preferable.

The content of the histidine or a salt thereof in the formulation of the present invention is preferably 0.1-3 wt %, more preferably 0.1-0.75 wt %, further preferably 0.1-0.2 wt %, and further more preferably 0.13 wt %, relative to the total amount of the formulation.

When the formulation of the present invention is a liquid formulation, the concentration of histidine or a salt thereof in the liquid formulation is preferably 5-200 mM, more preferably 5-50 mM, further preferably 5-15 mM, and further more preferably 10 mM. Here, histidine or a salt thereof is preferably used as a buffer containing histidine and histidine hydrochloride.

The formulation of the present invention may further contain saccharide. Examples of the saccharide include reducing sugar (e.g., glucose, fructose, maltose, lactose, arabinose, mannitol) and =reducing sugar (e.g., trehalose, sucrose). When saccharide is contained, the stability of the immunoconjugate of the formula (I) in the formulation is further improved (formation of immunoconjugate complex is suppressed etc.). The saccharide is preferably a nonreducing sugar, more preferably sucrose, trehalose, and further preferably sucrose.

The content of the saccharide in the formulation of the present invention is preferably 1-20 wt %, more preferably 3-15 wt %, further preferably 5-10 wt %, and further more preferably 7.5 wt %, relative to the total amount of the formulation.

When the formulation of the present invention is a liquid formulation, the content of saccharides in the liquid formulation is preferably 1-20% (w/v), more preferably 3-15% (w/v), further preferably 5-10% (w/v), and further more preferably 7.5% (w/v).

Preferable specific examples of the formulation of the present invention include a formulation comprising

(i) an immunoconjugate represented by the following formula (I-5):

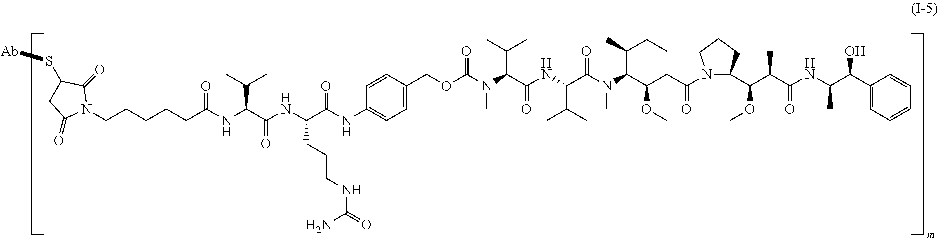

wherein Ab is an anti-GCC antibody molecule comprising a complementarity determining region defined by the following amino acid sequence:
light chain: CDR1 SEQ ID NO: 1
CDR2 SEQ ID NO: 2
CDR3 SEQ ID NO: 3
   heavy chain: CDR1 SEQ ID NO: 4
CDR2 SEQ ID NO: 5
CDR3 SEQ ID NO: 6, and
   m is an integer from 1-8,
   or a pharmaceutically acceptable salt thereof,
   (ii) 0.08% (w/v) of polysorbate 20,
   (iii) 10 mM of histidine or a salt thereof, and
   (iv) 7.5% (w/v) of sucrose,
   which has a pH within the range of 4.9-5.5.

The formulation of the present invention may further contain a pharmaceutically acceptable additive.

Examples of the pharmaceutically acceptable additive include carrier, excipient, buffering agent, isotonicity agent, dispersing medium, dispersion protector, non-ionic surfactant, surface activating agent, preservative, absorption retardant, pH adjuster and the like.

Examples of the dosage form of the formulation of the present invention include liquid formulation (e.g., solution formulation, dispersion formulation or suspension formulation, liposome formulation, and formulation obtained by re-dissolving freeze-dry formulation), semi-solid formulation (e.g., thick aqueous injection) and solid formulation (e.g., frozen liquid formulation, freeze-dry formulation).

As the formulation of the present invention, solution formulation, frozen liquid formulation, freeze-dry formulation or solution formulation obtained by re-dissolving freeze-dry formulation is preferable, and freeze-dry formulation is particularly preferable, from the aspects of stability and the like (e.g., suppression of decomposition of formulation components (e.g., immunoconjugate) and suppression of the development of byproducts (e.g., immunoconjugate complex)).

The formulation of the present invention can be produced by a method conventionally used in the technical field of formulation preparation by using immunoconjugate or a pharmaceutically acceptable salt thereof, polysorbate 20 and histidine or a salt thereof.

When the formulation of the present invention is a liquid formulation, the formulation of the present invention can be produced by dissolving, for example, immunoconjugate, polysorbate 20 and histidine or a salt thereof in distilled water for injection according to a method known per se.

When the formulation of the present invention is a frozen liquid formulation, the formulation of the present invention can be produced by freezing, for example, the above-mentioned liquid formulation according to a method known per se.

When the formulation of the present invention is a freeze-dry formulation, the formulation of the present invention can be produced by subjecting, for example, the above-mentioned liquid formulation to freeze-drying. The “freeze-drying” may be performed according to a method known per se and, for example, a method including freezing at −25° C. or below, and raising the temperature in the dryer to −25° C. to 40° C. while maintaining the degree of vacuum in the dryer at about 13.3 Pa or below can be used.

When the formulation of the present invention is a freeze-dry formulation, it preferably contains saccharides (e.g., nonreducing sugar (particularly, sucrose)) for stabilization and the like.

When the formulation of the present invention is a formulation obtained by re-dissolving a freeze-dry formulation, the formulation of the present invention can be produced by re-dissolving, for example, the above-mentioned freeze-dry formulation in an infusion or a solvent.

Examples of the “infusion” include electrolyte liquid, nutrient infusion and the like.

Examples of the “solvent” include water for injection (distilled water for injection), protein amino acid injection, vitamin injection, blood substitute combined with electrolytic solution and nutrient infusion (carbohydrate solution and the like), fat emulsion obtained by emulsifying fat, or a mixed solvent of two or more kinds of these. The solvent may contain a pH adjuster (e.g., acidic substance, weak alkaline substance) and the like where necessary.

The aforementioned “electrolyte liquid” is a liquid obtained by dissolving an electrolyte in water for injection. Examples thereof include a solution containing one or more kinds of sodium chloride, potassium chloride, calcium chloride, sodium lactate, sodium dihydrogen phosphate, magnesium carbonate and the like, lactated Ringer's solution, and acetated Ringer's solution. A preferable electrolyte liquid is a solution containing sodium chloride, particularly preferably physiological saline [0.9% (w/v) sodium chloride solution].

The aforementioned “carbohydrate solution” is a solution obtained by dissolving saccharides in water for injection. Examples thereof include a solution containing one or more kinds of glucose, fructose, sorbitol, mannitol, dextran and the like, and the like. A preferable carbohydrate solution is 5-700 (w/v) glucose solution, particularly preferably 5% (w/v) glucose solution, 10% (w/v) glucose solution and the like.

The aforementioned “protein amino acid injection” is a solution obtained by dissolving amino acid in water for injection. Examples thereof include a solution containing one or more kinds of glycine, aspartic acid, lysine and the like.

The aforementioned “vitamin injection” is a solution obtained by dissolving vitamins in water for injection. Examples thereof include a solution containing one or more kinds of vitamin B₁, vitamin C and the like.

As the infusion or solvent used for re-dissolving, water for injection, physiological saline or glucose solution (e.g., 5% (w/v) glucose solution) is preferable.

The amount of the aforementioned “infusion” or “solvent” to be used for one dosing is, for example, 5-1000 ml, preferably 5-500 ml.

The pH of the formulation of the present invention may be adjusted to suppress decomposition of formulation components (e.g., immunoconjugate) and suppress development of reaction byproducts (e.g., immunoconjugate complex)). From the aspects of stability of immunoconjugate in the formulation, the formulation of the present invention preferably has pH 4.5-7.0, more preferably pH 4.7-6.0, further preferably pH 4.9-5.5, further more preferably pH 5.2.

The formulation of the present invention provides a superior effect as a medicament. Since the formulation of the present invention shows low toxicity and less side effects, it is useful as a therapeutic or prophylaxis agent for, for example, cancer in mammals (e.g., human, bovine, horse, swine, dog, cat, monkey, mouse, rat, particularly human).

Examples of the cancer for which the formulation of the present invention is effective include cancers expressing GCC (e.g., gastrointestinal cancer (e.g., (1) colorectal cancer (e.g., colorectal glandular cancer, colorectal leiomyosarcoma, colorectal lymphoma, colorectal melanoma, or colorectal neuroendocrine tumor), (2) gastric cancer (e.g., stomach glandular cancer, stomach lymphoma, or stomach sarcoma), (3) esophagus cancer (e.g., esophagus flat epithelial cell cancer, esophagus glandular cancer, or cancer of the gastroesophageal junction), and (4) small intestine cancer), pancreatic cancer, lung cancer (e.g., squamous cell carcinoma or adenocarcinoma), soft tissue sarcomas (e.g., leiomyosarcoma or rhabdomyosarcoma), and neuroendocrine tumors (e.g., gastrointestinal or bronchopulmonary neuroendocrine tumors)).

In the present specification, being effective means, for example, to delay, interrupt, prevent or discontinue growth and/or metastasis of cancer cells in a test subject, and does not necessarily mean complete elimination of tumor growth.

While the formulation of the present invention can be safely administered orally or parenterally, it is preferably administered parenterally. Examples of the parenteral administration include intravenous administration, subcutaneous administration, abdominal administration, muscular administration and intravenous administration, with preference given to intravenous administration.

The formulation of the present invention is preferably an injection formulation, more preferably a formulation for intravenous injection.

While the dose of the formulation of the present invention varies depending on the subject of administration, administration frequency thereof and the like, the formulation shows effectiveness over the wide range. For example, the dose of the formulation of the present invention for an adult solid tumor patient (e.g., gastrointestinal cancer patient) in the amount of the immunoconjugate or a salt thereof contained in the formulation of the present invention is generally 0.05-50 mg/kg of the subject's body weight per dose, preferably 0.1-3.2 mg/kg of the subject's body weight per dose, more preferably 0.5-2.7 mg/kg of the subject's body weight per dose, even more preferably 1.0-2.0 mg/kg of the subject's body weight per dose, still more preferably 1.0-1.8 mg/kg of the subject's body weight per dose. When the formulation of the present invention is used in combination with other anti-cancer agent, the dose thereof may be smaller than the aforementioned dose. However, the dose of the formulation to be actually administered is determined according to various formulation forms, age, body weight and sex of the patient, disease level, administration route, term and interval of the administration, and the like, and can be altered at any time based on the judgment of the doctor.

The formulation of the present invention can be administered on a dosing schedule of once weekly, once every two weeks, once every three weeks, or once every four weeks. It is needless to say that the dosing period and frequency of the formulation of the present invention are changed depending on various situations, and are altered at any time based on the judgment of the doctor.

### EXAMPLES

The present invention is explained in more detail in the following by referring to Examples, which are not to be construed as limitative. In the following Examples and Comparative Examples, the formulation additives used were the Japanese Pharmacopoeia 16th Edition or Japanese Pharmaceutical Excipients 2003 compatible products.

### Reference Example 1

Preparation of Immunoconjugate a (5F9 vcMMAE)

Conjugation of vc and MMAE (Seattle Genetics, Inc., Bothell, Wash.), and conjugation of vcMMAE produced by the above-mentioned conjugation and antibody 5F9 could be performed by a method known per se (see, for example, US2006/0074008) or a method analogous thereto. In the present specification, immunoconjugate produced by the conjugation of vcMMAE and antibody 5F9 is also sometimes referred to as immunoconjugate A or 5F9 vcMMAE.

For example, conjugation of vcMMAE and 5F9 mAb could be performed as follows. First, 17.8 mg/mL 5F9 mAb solution in 100 mM acetate (pH 5.8) is adjusted with 0.3 M disodium phosphate to pH 8 to give the final 5F9 mAb concentration of 11.3 mg/ml. Then, DTPA is added to the reaction mixture to the final concentration of 1 mM. 5F9 mAb is added to 2.28 molar equivalents of tris(2-carboxyethyl)phosphine (relative to mole of 5F9 mAb) to partially reduce the mixture, and the mixture is stirred at 37° C. for 1.5 hr. The partially reduced 5F9 mAb solution is cooled to 4° C., and 4.4 mol equivalents of vcMMAE (relative to mole of antibody) in DMSO is added as a 20.3 mM solution. The mixture is stirred at 22° C. for 30 min, 5 mol equivalents of N-acetylcysteine (relative to mole of vcMMAE) is added and the mixture is further stirred for 15 min. Excess quenched vcMMAE and other reaction constituent components are removed by ultrafiltration/diafiltration of the immunoconjugate using 10 diafiltration volume of PBS (pH 7.4) to produce immunoconjugate A.

Immunoconjugate A is represented by the following formula (I-5)

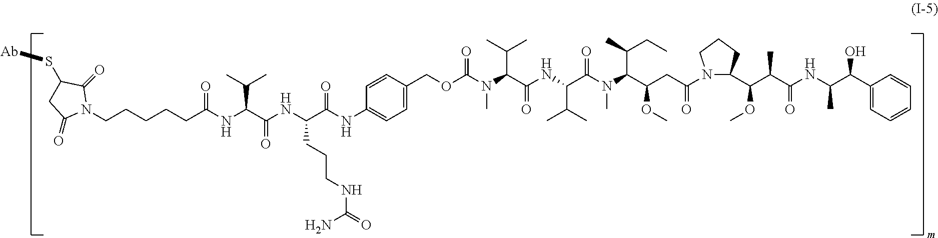

wherein Ab is 5F9 mAb, and m is 1-8.

By producing immunoconjugate based on the above-mentioned production method, for example, immunoconjugate having an average drug load (m) of about 3.6 could be produced.

### Example 1

A 30 mM histidine/histidine hydrochloride solution (pH 6) containing 8 mg/mL immunoconjugate A was prepared. The solution was dispensed by 0.3 mL to a glass vial (manufactured by Daiwa Special Glass co., ltd.) to prepare aliquot samples.

To the above-mentioned aliquot samples was added 10% (w/v) polysorbate 20 to a final concentration of 0.1% (w/v) to prepare polysorbate 20 addition samples.

### Comparative Example 1

A 30 mM citric acid/sodium citrate solution (pH 7) containing 8 mg/mL immunoconjugate A was prepared. The solution was dispensed by 0.3 mL to a glass vial (manufactured by Daiwa Special Glass co., ltd.) to prepare aliquot samples.

To the above-mentioned aliquot samples was added 10% (w/v) polysorbate 20 to a final concentration of 0.1% (w/v) to prepare polysorbate 20 addition samples.

### Experimental Example 1

Each sample described in Comparative Example 1 and Example 1 was preserved at 40° C. for 10 days and subjected to size-exclusion chromatography (SEC) using TSKgel G3000 SWXL column (manufactured by Tosoh), and the ratio of the monomer of the immunoconjugate A contained in the samples (SEC % monomer) was measured. The above-mentioned measurement results are shown in Table 1.

TABLE 1

| buffering agent | additive | SEC % monomer |
|---|---|---|
| citric acid | — | 89.89 |
| citric acid | 0.1% polysorbate 20 | 92.81 |
| histidine | — | 94.25 |
| histidine | 0.1% polysorbate 20 | 95.14 |

From Table 1, it has been found that a decrease in the monomer of immunoconjugate A is suppressed, and the stability of immunoconjugate A is improved by formulating a histidine buffering agent (histidine/histidine hydrochloride solution).

In addition, it was found that the effect of a histidine buffering agent to improve stability of immunoconjugate A can be observed even in the presence of polysorbate 20.

### Comparative Example 2

A 30 mM histidine/histidine hydrochloride solution (pH 6) containing 8 mg/mL immunoconjugate A was prepared. The solution was dispensed by 0.3 mL to a glass vial (manufactured by Daiwa Special Glass co., ltd.) to prepare aliquot samples.

### Example 2

To the aliquot samples described in Comparative Example 2 was added 10% (w/v) polysorbate 20 to a final concentration of 0.1% (w/v) to prepare polysorbate 20 addition samples.

### Comparative Example 3

To the aliquot samples described in Comparative Example 2 was added 10% (w/v) polysorbate 80 to a final concentration of 0.1% (w/v) to prepare polysorbate 80 addition samples.

### Experimental Example 2

Each sample described in Comparative Examples 2-3 and Example 2 was preserved at 40° C. for 10 days and subjected to size-exclusion chromatography (SEC) using TSKgel G3000 SWXL column (manufactured by Tosoh), and the ratio of the complex of the immunoconjugate A contained in the samples (SEC % complex) was measured. The above-mentioned measurement results are shown in Table 2.

TABLE 2

| buffering agent | additive | SEC % complex |
|---|---|---|
| histidine | — | 4.30 |
| histidine | polysorbate 20 | 3.38 |
| histidine | Polysorbate 80 | 5.29 |

From Table 2, it was found that the formation of immunoconjugate A complex can be suppressed by adding polysorbate 20. On the other hand, the formation of immunoconjugate A complex was not suppressed when polysorbate 80 was added.

### Example 3

A 10 mM histidine/histidine hydrochloride solution (pH 5) containing 25 mg/mL immunoconjugate A and 7.5% (w/v) sucrose was prepared. The solution was dispensed by 0.6 mL to a 3.5 mL glass vial (manufactured by Daiwa Special Glass co., ltd.) to prepare aliquot samples.

To the above-mentioned aliquot samples was added 10% (w/v) polysorbate 20 to a final concentration of 0.08% (w/v).

### Experimental Example 3

Stirrer chips were cast in the aliquot samples described in Example 3, and stirred at 800 rpm for 40 min. The turbidity of each sample was measured by a turbidimeter (TN-100, manufactured by EUTECH). The results of the above-mentioned measurement are shown in Table 3.

TABLE 3

| polysorbate 20 concentration | turbidity (NTU) |
|---|---|
| 0% (w/v) | 68.80 |
| 0.08% (w/v) | 24.16 |

From Table 3, it is confirmed that an increase in the turbidity during stirring of sample is suppressed, and the stability of immunoconjugate A against mechanical stress is improved by adding polysorbate 20.

A 10 mM histidine/histidine hydrochloride solution (pH 5.2) containing 25 mg/mL immunoconjugate A, 7.5% (w/v) sucrose and 0.08% (w/v) polysorbate 20 was prepared as a test sample and used in the following Experimental Examples 4-8. In Experimental Examples 4-8, the stability of test samples preserved under long-term preservation conditions (not more than −60° C. (sometimes to be referred to as “<−60° C.” in the present specification)) or stress conditions (temperature: 40° C.±2° C. and relative humidity (RH): 75%±5%) was studied.

### Experimental Example 4

The test samples (75 μg) preserved under long-term preservation conditions or stress conditions were subjected to a size-exclusion chromatography (SEC) analysis using phosphate-sodium chloride buffering system (pH 6.8) (specifically, analysis using silica-based porous bead column (tandem column)). In the above-mentioned analysis, the absorbance at wavelength 280 nm was monitored, and the ratio of the monomer and complex contained in the test samples was calculated from the above-mentioned absorbance. The results of the above-mentioned analysis are shown in Table 4.

TABLE 4

| preservation conditions; | | |
|---|---|---|
| preservation period | SEC % monomer | SEC % complex |
| ≤−60° C.; 0 month | 97 | 1.79 |
| 40° C./75% RH; 1 month | 91 | 5.86 |
| 40° C./75% RH; 2 months | 85 | 10.00 |
| ≤−60° C.; 2 months | 97 | 1.90 |

### Experimental Example 5

The test samples preserved under long-term preservation conditions or stress conditions were diluted with pure water and ampholytic electrolyte solution, and analyzed by imaged capillary isoelectric focusing (icIEF). The stability of the above-mentioned electrophoresis was confirmed by using hemoglobin as a control. In addition, pI of each peak of icIEF profile obtained by the above-mentioned analysis was calculated by normalizing with low and high pI markers (specifically, 8.18 and 10.10, respectively). The ratio of each peak was calculated from the area under the peak. The pI of major isoform detected from the test samples, the ratio of the major isoform in the test samples, and the ratios of acidic peak and basic peak are shown in Tables 5 and 6.

TABLE 5

| preservation conditions; | pI of major | ratio (%) of |
|---|---|---|
| preservation period | isoform | major isoform |
| ≤−60° C.; 0 month | 9.59 | 44.86 |
| 40° C./75% RH; 1 month | 9.57 | 28.36 |
| 40° C./75% RH; 2 months | 9.52 | 28.77 |
| ≤−60° C.; 2 months | 9.54 | 41.16 |

TABLE 6

| preservation conditions; | ratio (%) of | ratio (%) of |
|---|---|---|
| preservation period | acidic peak | basic peak |
| ≤−60° C.; 0 month | 27.18 | 27.97 |
| 40° C./75% RH; 1 month | 54.51 | 17.14 |
| 40° C./75% RH; 2 months | 46.99 | 24.24 |
| ≤−60° C.; 2 months | 29.92 | 28.93 |

### Experimental Example 6

The test samples preserved under long-term preservation conditions or stress conditions were analyzed by hydrophobic interaction chromatography (HIC), and drug loading distribution, an average molar ratio (DAR) of therapeutic agent forming a complex with antibody and the amount of ADC noncomplexed (free) antibody were determined. Specifically, the test samples after preservation were first diluted with dilution buffer, then with phosphate buffer and ammonium sulfate buffer, and subjected to HIC analysis using butyl-based nonporous bead column. In the above-mentioned analysis, the absorbance at wavelength 280 nm was monitored, and the ratio of each kind of molecule in the test sample was calculated based on the area under the curve of the obtained profile. In this analysis, an immunoconjugate wherein a therapeutic agent and an antibody molecule are bonded was eluted at a later retention time than an antibody molecule not bound by a therapeutic agent. The average molar ratio of the therapeutic agent contained in the immunoconjugate is shown in Table 7.

TABLE 7

| preservation | average molar ratio (DAR) of |
|---|---|
| conditions; | therapeutic agent contained in |
| preservation period | immunoconjugate |
| ≤−60° C.; 0 month | 3.9 |
| 40° C./75% RH; 1 month | 3.9 |
| 40° C./75% RH; 2 months | 3.9 |
| ≤−60° C.; 2 months | 3.9 |

### Experimental Example 7

The GCC binding activity of the test samples preserved under the long-term preservation conditions or stress conditions was measured by ELISA. To be specific, a 96 well plate coated with GCC fusion protein purified from CHO cell supernatant (guanylyl cyclase C (GCC) antigen) was blocked, and a dilution series of the test sample was added. After the above-mentioned addition, the plate was washed, and goat anti-human antibody covalent bonded with horseradish peroxidase (HRP) was added. After the above-mentioned addition, the plate was washed, and the color was developed by adding 3,3′,5,5′-tetramethylbenzidine (TMB) substrate. The signal level of the above-mentioned color development was directly proportional to the number of ADC molecules bonded thereto. The binding constant can be calculated by plotting the reference standard and the sample dilution concentration of the test sample after preservation against the intensity of the measured signals, and fit them to 4 parameter curves. The binding constant EC₅₀ values of the test samples preserved under the long-term preservation conditions or stress conditions are shown in Table 8 as a percentage of the reference standard (denoted as conjugation activity (%) in Table 8).

### Experimental Example 8

The cell proliferation inhibitory activity of the test samples preserved under the long-term preservation conditions or stress conditions was measured using a cell expressing the target antigen (specifically, HEK293 cell expressing GCC). HEK293 cells (parent and GCC transformant) were preserved in liquid nitrogen, and dissolved when used for each assay. The above-mentioned cell diluted in a growth medium was plated in a 96 well plate, a reference standard sample and a test sample were added to the 96 well plate, and incubated at 37° C. for 70±2 hr. After the above-mentioned incubation, an indicator of the surviving cell (specifically, Alamar Blue) was added to each well. The relative fluorescence unit (RFU) was measured on a fluorescence plate reader. The cell proliferation inhibitory activity can be calculated by plotting the reference standard and the sample dilution concentration against the intensity of the measured relative signals, and fit them to 4 parameter curves. The cell proliferation inhibitory activity (IC_{H}) of the test samples preserved under the long-term preservation conditions or stress conditions is shown in Table 8 as a percentage of the reference standard (denoted as cytotoxic activity (%) in Table 8).

TABLE 8

| preservation conditions; | conjugation | cytotoxic |
|---|---|---|
| preservation period | activity (%) | activity (%) |
| ≤−60° C.; 0 month | 100 | 85 |
| 40° C./75% RH; 1 month | 117 | 79 |
| 40° C./75% RH; 2 months | 85 | 109 |
| ≤−60° C.; 2 months | 93 | 113 |

The test samples preserved under the long-term preservation conditions did not show any significant change in each property studied in Experimental Examples 4-8 even after 2-month preservation.

The test samples preserved under the stress conditions did not show a large change in the conjugation activity and cytotoxic activity in Experimental Examples 7 and 8 even after 1- or 2-month preservation. Therefrom it is clear that the formulations subjected to each test were highly stable.

### INDUSTRIAL APPLICABILITY

The formulation of the present invention contains an immunoconjugate containing an anti-GCC antibody molecule and a therapeutic agent, and is useful as a prophylactic or therapeutic agent for gastrointestinal cancer and the like.

This application is based on the Japanese Patent Application No. 2014-090602 filed on Apr. 24, 2014, entire content of which is expressly incorporated by reference herein.

1. A formulation comprising
(i) an immunoconjugate of the following formula (I):
AbX-Z)ₘ (I)
wherein
Ab is an anti-GCC antibody molecule,
X is a linker component,
Z is a therapeutic agent, and
m is an integer from 1-15,
or a pharmaceutically acceptable salt thereof,
(ii) polysorbate 20, and
(iii) histidine or a salt thereof.

2. The formulation according to claim 1, comprising 0.08% (w/v) of polysorbate 20.

3. The formulation according to claim 1, comprising 10 mM of histidine or a salt thereof.

4. The formulation according to claim 1, further comprising a saccharide.

5. The formulation according to claim 4, wherein the saccharide is a non-reducing sugar.

6. The formulation according to claim 4, wherein the saccharide is sucrose.

7. The formulation according to claim 4, comprising 7.5% (w/v) of saccharide.

8. The formulation according to claim 1, comprising 25 mg/ml of the immunoconjugate.

9. The formulation according to claim 1, which has a pH within the range of 4.9-5.5.

10. The formulation according to claim 1, wherein the anti-GCC antibody molecule comprises complementarity determining regions defined by the following amino acid sequences,
light chain: CDR1 SEQ ID NO: 1
CDR2 SEQ ID NO: 2
CDR3 SEQ ID NO: 3
heavy chain: CDR1 SEQ ID NO: 4
CDR2 SEQ ID NO: 5
CDR3 SEQ ID NO: 6.

11. The formulation according to claim 1, wherein the anti-GCC antibody molecule comprises a light chain variable region defined by the amino acid sequence of SEQ ID NO: 7 and a heavy chain variable region defined by the amino acid sequence of SEQ ID NO: 8.

12. The formulation according to claim 1,
wherein X is -Ap-Wq-Yr-
wherein
A is a stretcher unit,
p is 0 or 1,
each W is independently an amino acid unit,
q is an integer from 0-12,
Y is a self-immolative spacer unit, and
r is an integer from 0-2.

13. The formulation according to claim 1, wherein Z is maytansine or auristatin.

14. The formulation according to claim 1, wherein Z is monomethylauristatin E.

15. The formulation according to claim 1, wherein m is an integer from 3-5.

16. The formulation according to claim 1, wherein the immunoconjugate is represented by the formula (I-5): 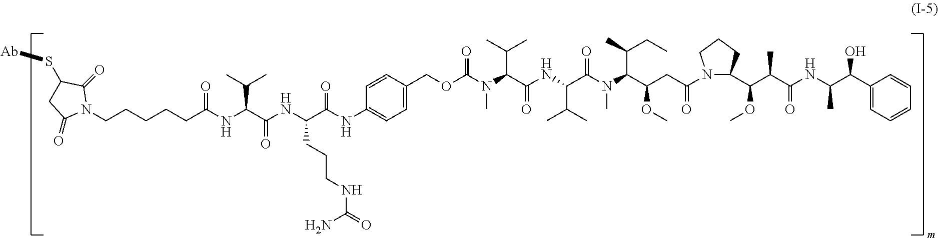
wherein
Ab is an anti-GCC antibody molecule, and
m is an integer from 1-15.

17. The formulation according to claim 1, which is an injection formulation.

18. The formulation according to claim 17, which is a formulation for intravenous injection.

19. The formulation according to claim 1, which is a liquid formulation.

20. The formulation according to claim 1, which is a frozen liquid formulation.

21. The formulation according to claim 1, which is a freeze-dry formulation.

22. A formulation comprising
(i) an immunoconjugate represented by the following formula (I-5): 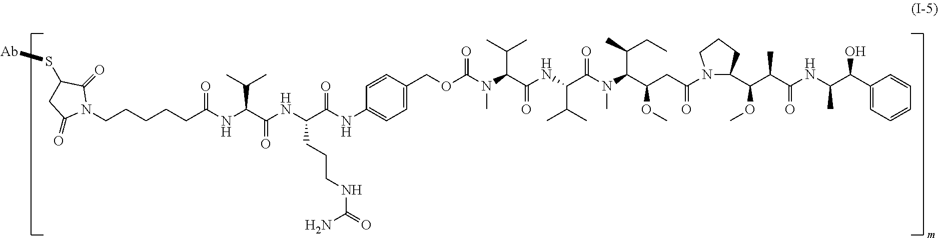
wherein
Ab is an anti-GCC antibody molecule comprising a complementarity determining region defined by the following amino acid sequence:
light chain: CDR1 SEQ ID NO: 1
CDR2 SEQ ID NO: 2
CDR3 SEQ ID NO: 3
heavy chain: CDR1 SEQ ID NO: 4
CDR2 SEQ ID NO: 5
CDR3 SEQ ID NO: 6, and
m is an integer from 1-8,
or a pharmaceutically acceptable salt thereof,
(ii) 0.08% (w/v) of polysorbate 20,
(iii) 10 mM of histidine or a salt thereof, and
(iv) 7.5% (w/v) of sucrose,
which has a pH within the range of 4.9-5.5.


SEQUENCE LISTING (10 sequences)

SEQ ID NO: 1 - protein, 11 aa, Artificial Sequence
  Description of Artificial Sequence Synthetic peptide
RASQSVSRNLA

SEQ ID NO: 2 - protein, 7 aa, Artificial Sequence
  Description of Artificial Sequence Synthetic peptide
GASTRAT

SEQ ID NO: 3 - protein, 9 aa, Artificial Sequence
  Description of Artificial Sequence Synthetic peptide
QQYKTWPRT

SEQ ID NO: 4 - protein, 5 aa, Artificial Sequence
  Description of Artificial Sequence Synthetic peptide
GYYWS

SEQ ID NO: 5 - protein, 16 aa, Artificial Sequence
  Description of Artificial Sequence Synthetic peptide
EINHRGNTNDNPSLKS

SEQ ID NO: 6 - protein, 11 aa, Artificial Sequence
  Description of Artificial Sequence Synthetic peptide
ERGYTYGNFDH

SEQ ID NO: 7 - protein, 107 aa, Artificial Sequence
  Description of Artificial Sequence Synthetic polypeptide
EIVMTQSPATLSVSPGERATLSCRASQSVSRNLAWYQQKPGQAPRLLIYGASTRATGIPARFSGSGSGTEFTLTIGSLQSEDFAVYYCQQYKTWPRTFGQGTNVEIK

SEQ ID NO: 8 - protein, 119 aa, Artificial Sequence
  Description of Artificial Sequence Synthetic polypeptide
QVQLQQWGAGLLKPSETLSLTCAVFGGSFSGYYWSWIRQPPGKGLEWIGEINHRGNTNDNPSLKSRVTISVDTSKNQFALKLSSVTAADTAVYYCARERGYTYGNFDHWGQGTLVTVSS

SEQ ID NO: 9 - protein, 233 aa, Artificial Sequence
  Description of Artificial Sequence Synthetic polypeptide
MGWSCIILFLVATATGVHSEIVMTQSPATLSVSPGERATLSCRASQSVSRNLAWYQQKPGQAPRLLIYGASTRATGIPARFSGSGSGTEFTLTIGSLQSEDFAVYYCQQYKTWPRTFGQGTNVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 468 aa, Artificial Sequence
  Description of Artificial Sequence Synthetic polypeptide
MGWSCIILFLVATATGVHSQVQLQQWGAGLLKPSETLSLTCAVFGGSFSGYYWSWIRQPPGKGLEWIGEINHRGNTNDNPSLKSRVTISVDTSKNQFALKLSSVTAADTAVYYCARERGYTYGNFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK